(19) United States
(12) Patent Application Publication
(10) Pub. No.: US 2006/0217619 A1
(43) Pub. Date: Sep. 28, 2006

(54) Heart state monitor method

(76) Inventor: Kuo-Yuan Chang, Shing-Tien City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/445,248

(22) Filed: Jun. 2, 2006

Related U.S. Application Data
(63) Continuation-in-part of application No. 10/345,485, filed on Jan. 17, 2003.

(51) Int. Cl. A61B 5/02

(52) U.S. Cl. 600/508

(57) ABSTRACT

A heart state monitor method is proposed. The pressure signal of a life body is measured and then performed by Fourier transform analysis. Next, the frequency domain data of the pressure signal are converted into a power spectral density (PSD). A heart beat noise index of the life body is then obtained according to the PSD. Finally, the heart state of the life body is diagnosed according to the heart beat noise index. The heart beat noise index may be quickly discriminated to let the life body and medical staffs grasp the current heart state of the life body at any time.

This is a continuation-in-part of application Ser. No. 10/345,485, filed Jan. 17, 2003.

### BACKGROUND OF THE INVENTION

1. Field of the Invention

The present invention relates to a heart state monitor method and, more particularly, to a monitor method capable of quickly and easily measuring and grasping the heart state of a life body at any time.

2. Background of the Related Art

Cardiovascular diseases are one of the primary pathogenic factors of people all the time. In addition to age, high blood fat, high cholesterol in the blood, hypertension, diabetes and heredity, people having a too high content of fat due to smoking, lack of exercise, fatness and abnormal diet may easily catch cardiovascular diseases. Cardiovascular diseases are basically measured by apparatuses. In the prior art, an electrocardiogram (ECG, EKG) of a heart is measured by an apparatus to determine which situation a patient suffers from. The electrocardiogram (ECG or EKG) is a noninvasive test that is used to reflect underlying heart conditions by measuring the electrical activity of the heart which records the electric voltage in the heart in the form of a continuous strip graph. ECG leads are attached to the body while the patient lies flat on a bed or table. Leads are attached to each extremity (4 total) and to 6 pre-defined positions on the front of the chest. A small amount of gel is applied to the skin, which allows the electrical impulses of the heart to be more easily transmitted to the ECG leads. The leads are attached by small suction cups, Velcro straps, or by small adhesive patches attached loosely to the skin. The test takes about 5 minutes and is painless. However, such variation like the arrhythmia or coronary disease occurs only when or after people take exercise. This kind of inspection is called an exercise electrocardiogram. Besides, because the arrhythmia or coronary disease may occur at any time in a day, an apparatus is taken along the patient all the day for grasping data momentarily. This kind of inspection is called an all-day electrocardiogram. However, either exercise electrocardiogram or the all-day electrocardiogram is very cumbersome for the patient. Moreover, because the cardiovascular diseases do not occur all the time, it is sometimes difficult to find them. Many people may have cardiovascular diseases and be unaware, hence allowing cardiovascular diseases to become silent killers of otherwise healthy individuals.

Beside the above drawbacks, when using a conventional measurement apparatus to measure the heart state, the patient needs to take off his/her clothes so that sticking sheets of the measurement apparatus can adhere to the vicinity of the heart for obtaining an electrocardiogram. This is very inconvenient for the patient, especially for a female patient. Moreover, the measured result of a conventional heart measurement apparatus can only be analyzed by medical staffs. Once the body of a patient has a bad status when he/she goes out for work or play, because the medical staffs cannot grasp the physiologic status of the patient immediately, it is usually too late for the patient to take medical treatment.

Accordingly, the present invention aims to propose a heart state monitor method to let a patient be able to measure and determine the health state of his heart at any time himself so as to resolve the problems in the prior art.

### SUMMARY OF THE INVENTION

One object of the present invention is to propose a heart state monitor method. Different from recording the electric voltage in the heart, the heart state moinitor device provided by the present invention measures pressure signals reflected from a heart by imposing one or more pressure values on one's arm, wrist, finger or ankle to accurately know the heart status of the life body after numerical processing. Instead of the adjustment of ECG by professional people, the life body may be easily measured by a man or woman and the information for the health state of his or her heart may be easily read out.

Another object of the present invention is to propose a heart state monitor method, which can transmit the measured pressure signal of a life body to a medical center via a transmission circuit, and can record and analyze the current heart state of the life body in real time. The current heart state of the life body can thus be easily measured and accurately recorded at any place so that the life body and medical staffs can grasp the current heart state of the life body and thereby make real-time and effective treatment.

To achieve the above objects, the present invention comprises the following steps. First, a pressure signal of a life body is measured. The pressure signal is transformed by means of Fourier transform and then converted into a power spectral density (PSD). A heart beat noise index of the life body is then obtained according to the PSD. Finally, the heart state of the life body is diagnosed according to the heart beat noise index.

Another embodiment of the present invention comprises the following steps. First, a pressure signal of a life body is measured. The pressure signal is then analyzed to obtain envelope functions thereof. Next, a roughness integral (Ri) and a roughness differential (Rd) are calculated out according to the envelope functions. Finally, the heart state of the life body is diagnosed according to the roughness integral and the roughness differential.

The various objects and advantages of the present invention will be more readily understood from the following detailed description when read in conjunction with the appended drawings, in which:

### BRIEF DESCRIPTION OF THE DRAWINGS

FIG. 1 shows a flowchart of the present invention;

FIG. 2A shows the Fourier transform of the heart beat signal of a testee without any heart disease;

FIG. 2B is a power spectral density graph of FIG. 2A;

FIG. 3 is a power spectral density graph of a patient with Atrial Fib. (AF);

FIG. 4 is a power spectral density graph of a patient with entricular (PVC);

FIG. 5 is a power spectral density graph of a patient with Atrial (PAC);

FIG. 6A shows envelopes of the heart beat signal of a testee without any heart disease;

FIG. 6B shows the area enclosed by the envelopes in FIG. 6A after time-domain integral;

FIG. 7A shows envelopes of the heart beat signal of a patient with cardiomyopathy; and

FIG. 7B shows the area enclosed by the envelopes in FIG. 7A after time-domain integral.

### DETAILED DESCRIPTION OF THE INVENTION

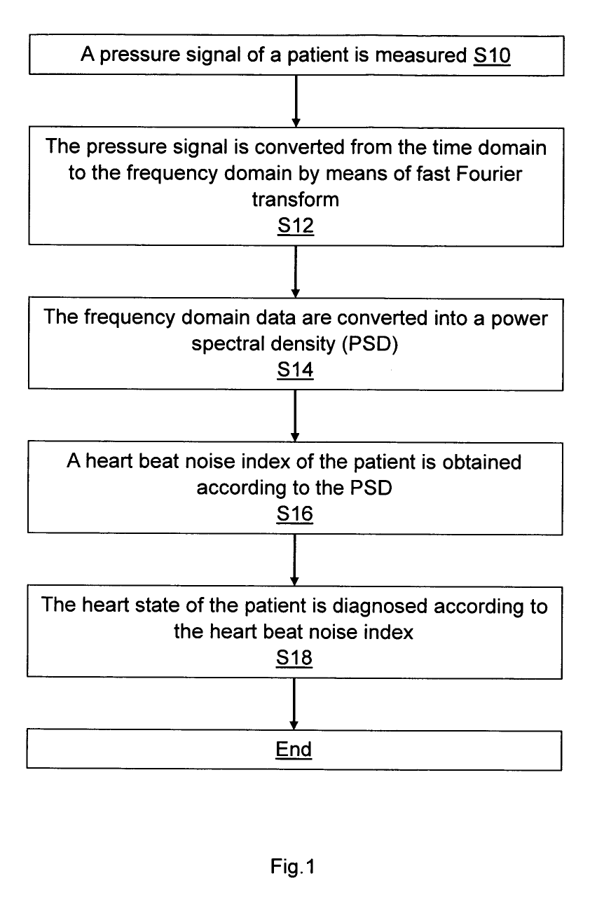

As shown in FIG. 1, a heart state monitor method of the present invention comprises the following steps. First, pressure is imposed on the arm of a patient for measurement to obtain a pressure signal that represents blood injected into a main artery by heart (Step S10). The pressure signal is then transmitted to a medical center via a transmission circuit. The medical center transforms the received pressure signal from the time domain to the frequency domain by means of Fourier transform, and then integrates and stores these data into a database (Step S12). The frequency domain data are then converted into a power spectral density (PSD) (Step S14). Next, a heart beat noise index of the patient is calculated out according to many noise waveforms between harmonics in the PSD (Step S16). Finally, the heart state of the patient is diagnosed according to the heart beat noise index (Step S18).

Accordingly, the pressure signal is acquired by imposing pressure or pressures on a measured position for about 30 seconds. It is noted that heart may have abnormal condition at intervals of 15 or more seconds for some people. For conventional ECG measurement, detection may last about or less 10 seconds, which is not enough for some people at the risk of heart diseases. Thus, the present invention provides a measure method enable to acquire the information more representing heart state at the risk of heart diseases. Moreover, it is advantageous for heart to be detected by pressure. Furthermore, normal people without professional knowledge on ECG may operate the heart state monitor without the utilization of any additional probing equipment. In addition, the pressure may be imposed on other measured positions, such as a wrist, finger or ankle of a patient for acquiring the pressure signal of a heart. Or an infrared physiology measurement device can further be utilized for measurement. Besides, the transmission circuit may be a telephone circuit, an integrated service digital network (ISDN), an asymmetrical digital subscriber loop (ADSL), a high-speed digital subscriber loop (HDSL), a transmission control protocol/Internet protocol (TCP/IP), a cable circuit, an infrared transmission circuit or a radio-frequency (RF) transmission circuit.

In addition to measuring the physiologic state of heart by a patient himself, the present invention can also transmit the patient's pressure signal to a medical center for analysis. Therefore, no matter the patient is at home or goes out for work or playing, he can perform measurement and then transmit the measured pressure signal to the location of a medical center or medical staffs if necessary to let them grasp his status at any time. Additionally, the pressure signal of the patient can also be transmitted to any medical equipment or device for analysis and storage of the pressure signal.

The present invention is cooperated with National Taiwan University, ROC for research and development, and Cathay General Hospital, ROC is then entrusted for experiments. First, a testee without any heart disease is measured with the above method to obtain heart beat signal data of this testee. The data are heart beat signals with respect to time as shown in the table below:
150.5829 317.6681 −447.623 22.69058 20.6278 105.2018 51.5695 78.38564 18.56502 655.9641 −447.623 33.00448 6.18834 113.4529 55.69506 47.44394 26.81614 606.4573 −402.242 37.13004 4.12556 84.57398 59.82062 30.9417 66.00896 198.0269 −200.09 43.31838 −2.06278 39.19282 61.8834 41.2556 24.75336 −257.848 −88.6995 121.704 2.06278 24.75336 59.82062 49.50672 6.18834 −447.623 −39.1928 99.01344 16.50224 30.9417 72.1973 47.44394 18.56502 −307.354 −2.06278 55.69506 22.69058 33.00448 129.9551 45.38116 22.69058 −80.4484 6.18834 24.75336 72.1973 30.9417 88.69954 39.19282 20.6278 −18.565 18.56502 22.69058 138.2063 30.9417 45.38116 43.31838 18.56502 41.2556 30.9417 16.50224 99.01344 30.9417 22.69058 66.00896 24.75336 −16.5022 35.06726 12.37668 59.82062 28.87892 24.75336 105.2018 24.75336 12.37668 74.26008 14.43946 18.56502 43.31838 33.00448 80.44842 20.6278 12.37668 94.88788 37.13004 18.56502 115.5157 33.00448 59.82062 57.75784 14.43946 45.38116 49.50672 26.81614 96.95066 30.9417 41.2556 76.32286 20.6278 6.18834 57.75784 24.75336 63.94618 30.9417 51.5695 14.43946 26.81614 −6.18834 125.8296 22.69058 35.06726 28.87892 53.63228 4.12556 28.87892 12.37668 152.6457 24.75336 35.06726 30.9417 51.5695 12.37668 30.9417 28.87892 105.2018 26.81614 41.255699.01344 43.31838 26.81614 66.0089624.75336 68.07174 43.31838 35.06726 101.076243.31838 22.69058 82.511224.75336 33.00448 134.0807 35.06726 72.1973 43.31838 22.69058 35.06726 30.9417 6.18834 111.3901 30.9417 37.13004 66.00896 24.75336 −14.4395 28.87892 −6.18834 70.13452 33.00448 26.81614 86.63676 28.87892 3.81E-07 59.82062 −4.12556 26.81614 43.31838 37.13004 68.07174 24.75336 24.75336 125.8296 4.12556 24.75336 55.69506 43.31838 45.38116 35.06726 30.9417 72.1973 18.56502 33.00448 111.3901 39.19282 39.19282 84.57398 33.00448 37.13004 28.87892 37.13004 84.57398 33.00448 47.44394 68.07174 226.9058 4.12556 96.95066 26.81614 51.5695 33.00448 53.63228 16.50224 996.3228 2.06278 129.9551 30.9417 24.75336 37.13004 49.50672 −8.25112 1014.8886.18834 94.88788 24.75336 37.13004 68.0717449.50672 39.19282 253.7219 16.50224 53.63228 33.00448 51.5695 111.3901 51.5695 51.5695 45.38116 30.9417 84.57398 47.44394 63.9462 66.00896 82.5112 49.50672 49.50672 94.88788 49.50672 59.8206 63.94618 84.57398 49.50672 70.13452 78.38564 49.50672 55.6951 68.07174 63.94618 63.94618 59.82062 63.94618 72.1973 66.009 68.07174 43.31838 80.44842 57.75784 63.94618 72.1973 66.009 72.1973 49.50672 66.00896 61.8834 76.32286 72.1973 84.574 84.57398 57.75784 53.63228 51.5695 72.1973 61.8834 84.574 103.139 66.00896 47.44394 63.94618 57.75784 59.82062 88.6995 109.3273 63.94618 53.63228 80.44842 51.5695 57.75784 88.6995 94.88788 63.94618 59.82062 66.00896 45.38116 55.69506 99.0134 76.32286 66.00896 61.8834 51.5695 45.38116 55.69506 80.4484 61.8834 70.13452 59.82062 51.5695 51.5695 59.82062 66.009 53.63228 105.2018 74.26008 51.5695 70.13452 61.8834 53.6323 53.63228 96.95066 86.63676 51.5695 63.94618 63.94618 51.5695 53.63228 90.76232 92.8251 53.63228 45.38116 76.32286 57.7578 57.75784 72.1973 82.5112 49.50672 47.44394 68.07174 70.1345 61.8834 78.38564 57.75784 49.50672 53.63228 61.8834 70.1345 55.69506 70.13452 45.38116 63.94618 53.63228 53.63228 57.7578 74.26008 53.63228 22.69058 66.00896 55.69506 61.8834 55.6951 68.07174 39.19282 45.38116 80.44842 59.82062 63.94618 59.8206 55.69506 41.2556 49.50672 72.1973 68.07174 61.8834 63.94618 66.00896 43.31838 49.50672 59.82062 84.57398 61.8834 57.75784 61.8834 53.63228 59.82062 49.50672 113.4529 59.82062 53.63228 72.1973 76.32286 68.07174 53.63228 136.1435 63.94618 55.69506 63.94618 61.8834 53.63228 61.8834 113.4529 74.26008 51.5695 68.07174 51.5695 59.82062 66.00896 88.69954 84.57398 66.00896 70.13452 43.31838 59.82062 63.94618 55.69506 70.13452 76.32286 68.07174 51.5695 76.32286 68.07174 47.44394 66.00896 72.1973 74.26008 51.5695 63.94618 66.00896 49.50672 59.82062 68.07174 99.01344 49.50672 49.50672 70.13452 45.38116 63.94618 59.82062 76.32286

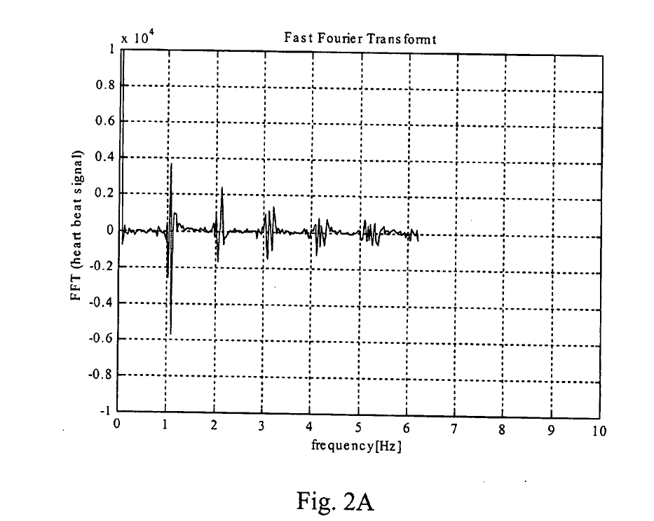

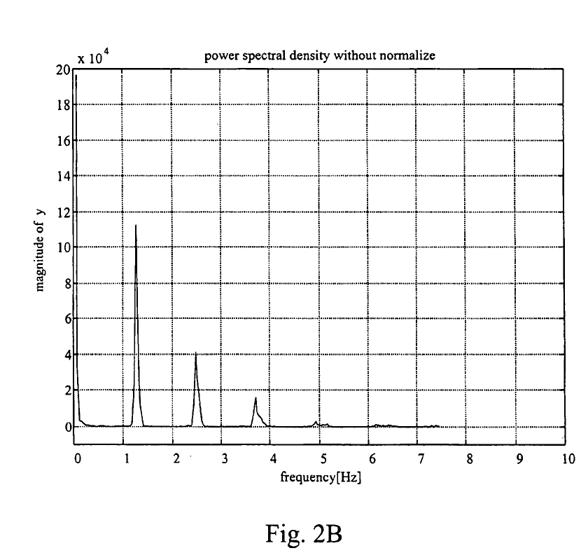

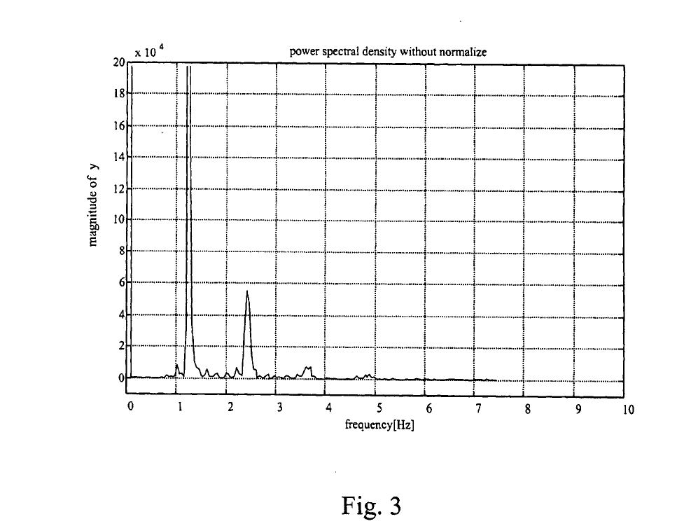

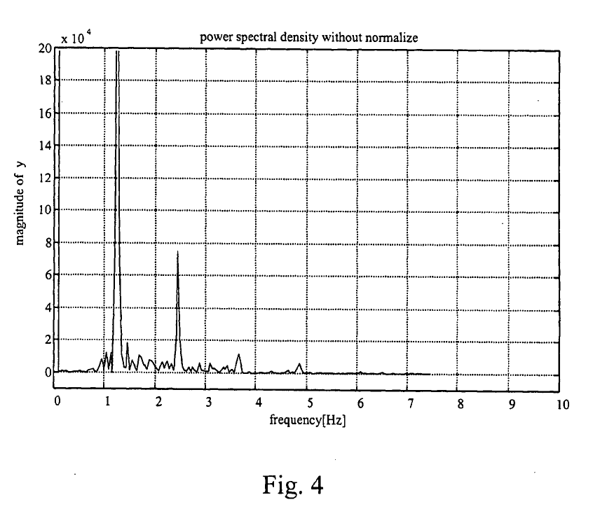

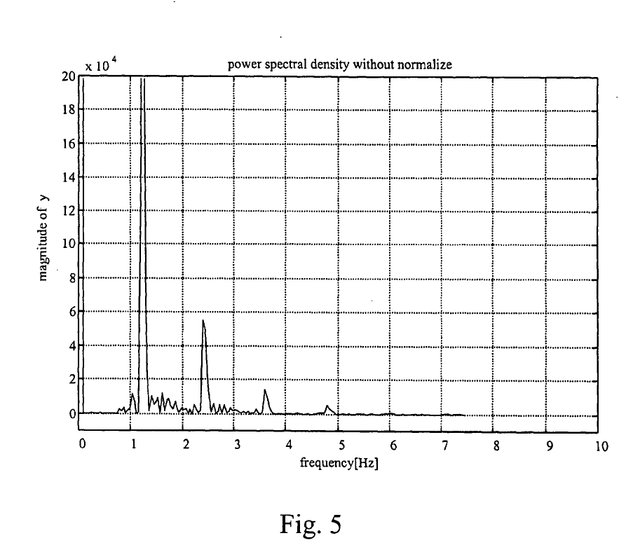

Next, the heart beat signal data in the table are converted from the time domain to the frequency domain by means of Fourier transform to obtain the frequency domain data shown in FIG. 2A. Finally, the frequency domain data shown in FIG. 2A are converted into a power spectral density (PSD) graph shown in FIG. 2B. As can be known from this figure, a flat waveform exists between each harmonic when the heart has no disease. However, there exist many noise waveforms between harmonics in the PSD graph if the heart has a disease. As shown in FIG. 3, the number of noise waveforms between harmonics in the PSD graph of a patient having Atrial Fib. (AF) is larger than that of a normal person. As shown in FIG. 4, the number of noise waveforms between harmonics in the PSD graph of a patient having entricular (PVC) is larger than that of a patient having Atrial Fib.(AF). As shown in FIG. 5, the number of noise waveforms between harmonics in the PSD graph of a patient having Atrial (PAC) is larger than that of a patient having entricular (PVC). From FIGS. 2B, 3, 4 and 5, we can know that the number of noise waveforms between each harmonic increases with the order of severity of the heart state. Therefore, noise waveforms between each harmonic can be utilized to calculate out a heart beat noise index of each patient. The health status of a patient can thus be determined according to the heart beat noise index. Accuracy and practicability of the present invention have been verified by cooperation with National Taiwan University, ROC for two years and experiments of Cathay General Hospital, ROC.

In addition to converting heart beat signals into a PSD for calculation and analysis of the heart beat noise index of a patient, the heart beat signal curve can also undergo an analysis of envelope function. This embodiment of the present invention comprises the following steps. First, a pressure signal like the heart beat signal of a patient is measured. The pressure signal is then analyzed to obtain envelope functions thereof. Next, a roughness integral (Ri) and a roughness differential (Rd) are calculated out according to the envelope functions. Finally, the heart state of the patient is diagnosed according to the roughness integral and the roughness differential.

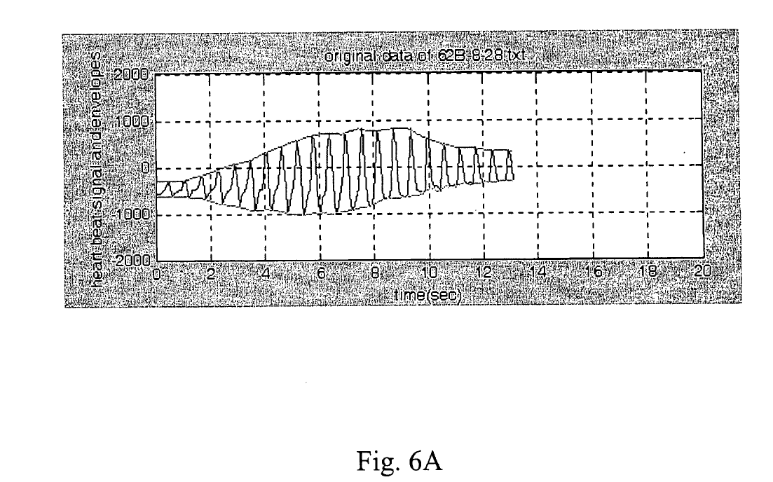

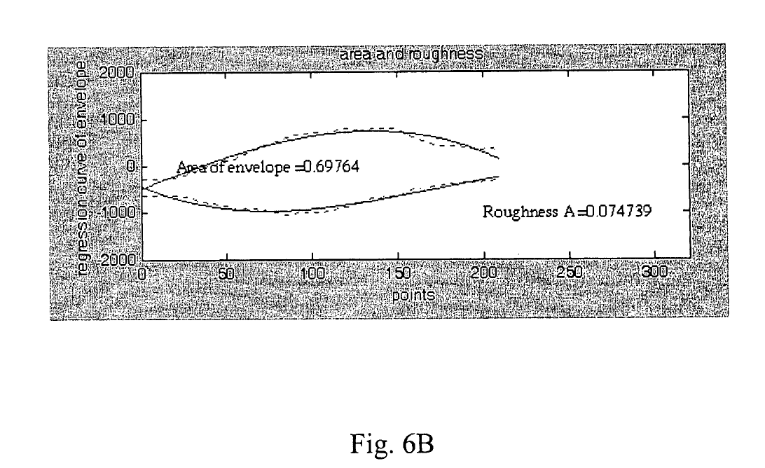

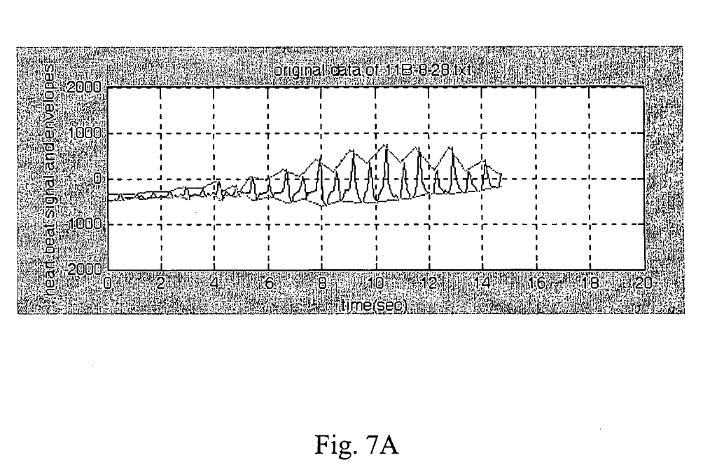

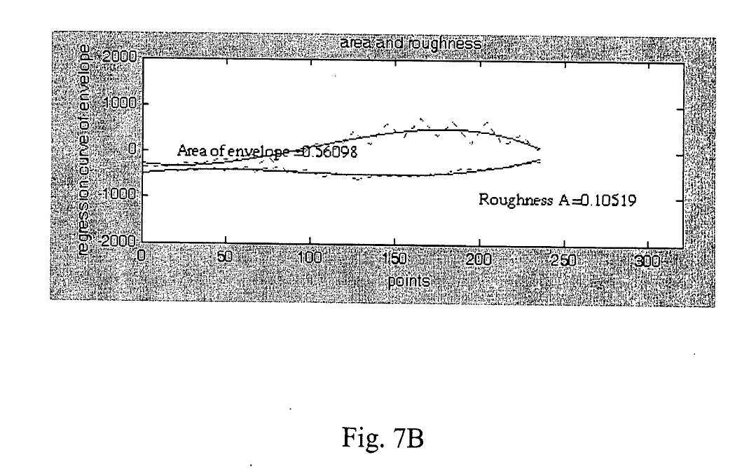

FIG. 6A shows envelopes of heart beat signal data of a testee without any heart disease. The area enclosed by the envelope of FIG. 6A is then integrated to obtain the area enclosed by the envelope and calculate out a roughness integral (Ri), as shown in FIG. 6B. The Ri of the testee without any heart disease is 0.074739. Next, FIG. 7A shows envelopes of heart beat signal data of a patient with cardiomyopathy. The area enclosed by the envelope of FIG. 7A is then integrated to obtain the area enclosed by the envelope and calculate out a roughness integral (Ri), as shown in FIG. 7B. The Ri of the patient with cardiomyopathy is 0.10519. Through comparison, it is evident the Ri of the patient with heart disease is larger than that of the testee without any heart disease. Cathay General Hospital, ROC measured many testees without any heart disease and many patients with valve disease or cardiomyopathy to obtain the following table:

2 Integral of Integral of File normalized File normalized Type name area Ri Type name area Ri Healthy 9-8-28 0.72696 0.04032 Valve disease 8-8-28 0.72859 0.05797 heart 21-8-28 0.70771 0.04595 54-8-28 0.71496 0.19529 25-8-28 0.72304 0.06440 78-8-28 0.66857 0.13034 26-8-28 0.73554 0.06750 4-8-30 0.77257 0.07005 33-8-28 0.70940 0.12065 15-8-30 0.69496 0.15187 52-8-28 0.71541 0.07768 20-8-30 0.65289 0.27694 67-8-28 0.75890 0.06634 31-8-30 0.64088 0.23327 72-8-28 0.75650 0.06634 64-8-30 0.65289 0.27694 95-8-28 0.77309 0.03792 44-94 0.73418 0.04754 99-8-28 0.72481 0.078335 64-9-4 0.65289 0.27694 5-8-30 0.74556 0.06534 68-94 0.59276 0.15297 12-8-30 0.70205 0.15187 70-9-4 0.81891 0.12632 18-8-30 0.65404 0.13052 Mean 0.69375 0.16637 23-8-30 0.67276 0.13320 Standard deviation 0.06311 0.08551 32-8-30 0.73545 0.08168 Cardiomyopathy 5-8-28 0.66557 0.14648 48-8-30 0.78127 0.04743 11-8-28 0.56098 0.10519 50-8-28 0.69595 0.08872 13-8-28 0.71496 0.19529 59-8-30 0.71071 0.09931 3-8-30 0.71364 0.07890 7-94 0.73095 0.06975 21-8-30 0.66731 0.23981 18-94 0.72239 0.07027 37-8-30 0.63333 0.23693 22-9-4 0.70103 0.04272 57-8-30 0.70211 0.28642 53-94 0.74003 0.12303 80-8-30 0.64971 0.10831 56-94 0.72689 0.09979 15-94 0.65891 0.18309 60-9-4 0.71768 0.05073 19-9-4 0.63151 0.22443 61-9-4 0.76758 0.07225 21-94 0.71839 0.19035 Mean 0.72543 0.07968 Mean 0.66513 0.18138 Standard deviation 0.03071 0.032996 Standard deviation 0.04723 0.06521

As can be known from the table, the mean of the roughness integral Ri of healthy hearts is 0.07968, the mean of the roughness integral Ri of hearts with valve disease is 0.16637, and the mean of the roughness integral Ri of hearts with is cardiomyopathy is 0.18138. Similarly, it is evident the Ri of the patients with heart disease is larger than that of the testees without any heart disease. Therefore, heart diseases like valve disease and cardiomyopathy of a patient can be determined based on the Ri value of his heart.

Moreover, the envelope function of the heart beat signals of a patient can be differentiated to obtain the roughness differential. Similarly, heart diseases like valve disease and cardiomyopathy of a patient can be determined based on the magnitude of the Rd value of his heart.

To sum up, the present invention proposes a heart state monitor method, which makes use of heart beat measurement of a life body at the arm, wrist, finger or ankle to obtain a heart beat noise index after numerical processing so as to accurately know the heart status of the life body without the inconvenience of taking off clothes. The life body can thus easily perform measurement and accurately obtain the health status of heart. Moreover, the present invention can transmit the measured heart pressure signal of a life body to a medical center via a transmission circuit, and can record and analyze the current heart state of the life body in real time. The life body can thus easily measure and accurately record the current heart state at any place to let the life body and medical staffs grasp the current heart state of the life body and make real-time and effective treatment.

Although the present invention has been described with reference to the preferred embodiments thereof, it will be understood that the invention is not limited to the details thereof. Various substitutions and modifications have been suggested in the foregoing description, and other will occur to those of ordinary skill in the art. Therefore, all such substitutions and modifications are intended to be embraced within the scope of the invention as defined in the appended claims.

What is claimed is:

1. A heart state monitor method comprising the steps of:
(a) measuring a pressure signal by imposing a pressure on a position of a life body;
(b) converting said pressure signal from a time domain to a frequency domain by means of Fourier transform and then converting said frequency domain data into a power spectral density;
(c) obtaining a heart beat noise index of said life body according to said power spectral density; and
(d) diagnosing the heart state of said life body according to said heart beat noise index.

2. The heart state monitor method as claimed in claim 1, wherein said position is an arm, wrist, finger or ankle of said life body.

3. The heart state monitor method as claimed in claim 1 further comprising a step of transmitting said pressure signal to a medical center via a transmission circuit for performing said Step (b) after said step (a).

4. The heart state monitor method as claimed in claim 3, wherein said transmission circuit is a telephone circuit, an ISDN, an ADSL, a HDSL, a TCP/IP, a cable circuit, an infrared transmission circuit or an radio-frequency transmission circuit.

5. The heart state monitor method as claimed in claim 1, wherein many noise waveforms between each harmonic in said power spectral density is further used to calculate out said heart beat noise index of said life body.

6. The heart state monitor method as claimed in claim 1, wherein said pressure signal of said life body before analysis and said frequency domain data and said power spectral density after analysis is further stored into a database.

7. A heart state monitor method comprising the steps of:
(a) measuring a pressure signal by imposing a pressure on a position of a life body;
(b) analyzing said pressure signal to obtain envelope functions thereof;
(c) calculating a roughness integral and a roughness differential according to said envelope functions; and
(d) diagnosing the heart state of said life body according to said roughness integral and said roughness differential.

8. The heart state monitor method as claimed in claim 7, wherein said position is an arm, wrist, finger or ankle of said life body.
